(19) United States
(12) Patent Application Publication
(10) Pub. No.: US 2023/0413659 A1
(43) Pub. Date: Dec. 21, 2023

(54) POLYMER

(71) Applicant: Sumitomo Chemical Co., Ltd., Tokyo (JP)

(72) Inventors: Nir Yaacobi-Gross, Godmanchester (GB); kIRAN KAMTEKAR, Godmanchester (GB)

(73) Assignee: Sumitomo Chemical Co., Ltd., Tokyo (JP)

(21) Appl. No.: 18/034,799

(22) PCT Filed: Oct. 29, 2021
(86) PCT No.: PCT/EP2021/080222

§ 371 Date: May 1, 2023

(30) Foreign Application Priority Data
Nov. 2, 2020 (GB) 2017341.5

(51) Int. Cl. H10K 85/10; C08G 61/12; H10K 30/30

(52) U.S. Cl. CPC H10K 85/113; C08G 61/126; H10K 85/151; H10K 30/30; C08G 2261/124; C08G 2261/1414; C08G 2261/3223; C08G 2261/3246; C08G 2261/3247; C08G 2261/3243; C08G 2261/411; C08G 2261/91

(57) ABSTRACT

A polymer comprising a repeating structure of formula (I): -D-X¹-A-X²-. D is a conjugated electron-donating group of formula (II); A is a conjugated electron-accepting group; X¹ and X² are each independently a conjugated bridge group selected from phenylene, thiophene, furan, thienothiophene, furofuran, thienofuran, thiazole, oxazole, alkene, alkyne and imine, each of which may be unsubstituted or substituted with one or more substituents. The polymer has a highest occupied molecular orbital (HOMO) level as measured by square wave voltammetry of no more than 5.30 eV from vacuum level. The polymer may be used as an electron donor in an organic photodetector.

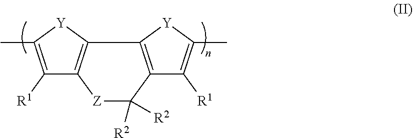

### BACKGROUND

Donor-acceptor (D-A) polymers are known for use in organic photovoltaic devices. Wang et al, “Effects of π-Conjugated Bridges on Photovoltaic Properties of Donor-π-Acceptor Conjugated Copolymers”, Macromolecules 2012, 45, 3, 1208-1216 discloses D-A polymers containing a 7r-bridge for use in photovoltaic devices.

Wang et al, “The effect of conjugated π-bridge and fluorination on the properties of asymmetric-building-block-containing polymers (ABC polymers) based on dithienopyran donor and benzothiadiazole acceptors” discloses a photovoltaic device containing a polymer in which hexylthiophene is inserted into D-A-type polymers based on asymmetric dithieno[3,2-b:2′,3′-d]pyran donor and benzothiadiazole, mono-fluorinated benzothiadiazole or di-fluorinated benzothiadiazole acceptor.

Putri et al, “Step-by-step improvement in photovoltaic properties of fluorinated quinoxaline-based low-band-gap polymers”, Organic Electronics, Volume 47, August 2017, Pages 14-23 discloses a solar cell containing a polymer having electron-donating dialkoxy-substituted benzodithiophene connected to electron-withdrawing 2,3-diphenylquinoxaline acceptor through a thiophene bridge.

WO 2018/039347 discloses polymers incorporating exocyclic cross-conjugated donors or substituents.

EP2767553 discloses a polymer comprising a constituent unit represented by Formula (1) and a constituent unit represented by Formula (2):

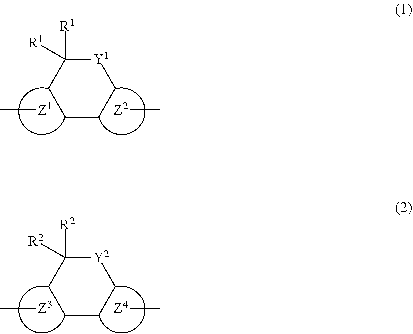

WO 2014/202184 discloses a polymer comprising a unit of formula T:

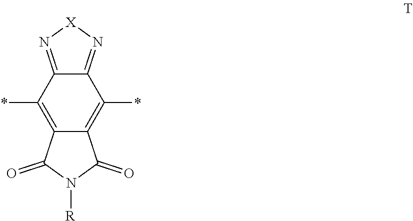

KR20160043858 discloses a polymer comprising a first unit of formula 1, a second unit of formula 2 or 3 and a third unit different from formulae 1-3:

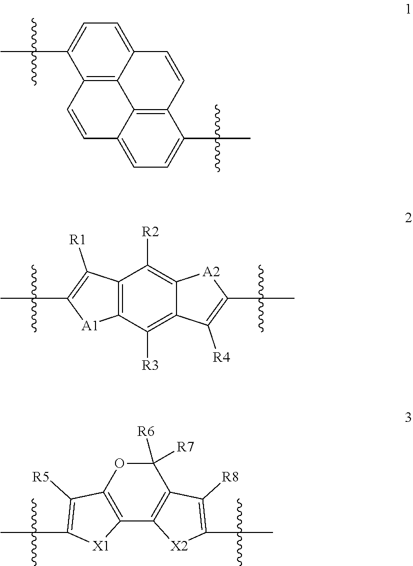

CN110776621 discloses a polymer of formula:

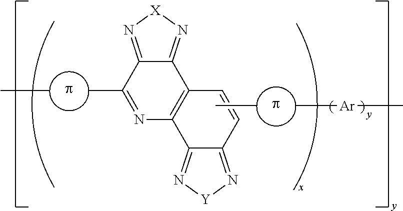

### SUMMARY

The present disclosure provides a polymer comprising a repeating structure of formula (I):

-D-X¹-A-X² (I)

wherein:
D is a conjugated electron-donating group of formula (II); A is a conjugated electron-accepting group; X¹ and X² are each independently a conjugated bridge group selected from phenylene, thiophene, furan, thienothiophene, furofuran, thienofuran, thiazole, oxazole, alkene, alkyne and imine, each of which may be unsubstituted or substituted with one or more substituents:

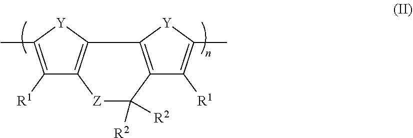

wherein:
Y in each occurrence is independently O or S;
Z is O, S or NR³ wherein R³ is H or a substituent;
R¹ in each occurrence is independently H or a substituent;
R² in each occurrence is independently a substituent,
n is at least 1; and
   wherein the polymer has a highest occupied molecular orbital (HOMO) level as measured by square wave voltammetry of no more than 5.30 eV from vacuum level, and/or wherein a model of the polymer of formula H-[-D-X¹-A-X²]₂-A has a highest occupied molecular orbital (HOMO) level as modeled using Gaussian09 with B3LYP (functional) and 6-31G (Basis set) of no more than 4.50 eV from vacuum level.

Optionally, the polymer has a HOMO level of no more than 5.10 eV from vacuum level as measured by square wave voltammetry.

Optionally the polymer has a HOMO level of at least 4.90, optionally at least 5.00 eV from vacuum level as measured by square wave voltammetry.

Optionally, the electron-accepting repeat unit is selected from formulae (Va) and (Vb):

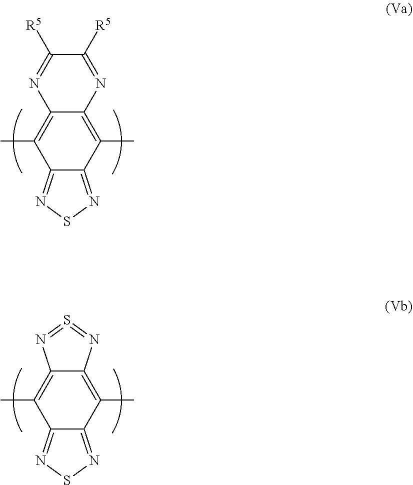

wherein R⁵ in each occurrence is H or a substituent.

Optionally, each R¹ is H.

Optionally, each R² is independently selected from the group consisting of:

linear, branched or cyclic C₁₋₂₀ alkyl wherein one or more non-adjacent C atoms may be replaced by O, S, NR⁸ , CO or COO wherein R⁸ is a C₁₋₁₂ hydrocarbyl and one or more H atoms of the C₁₋₂₀ alkyl may be replaced with F;
and
a group of formula (Ak)u-(Ar⁴)v wherein Ak is a C₁₋₁₂ alkylene chain in which one or more C atoms may be replaced with O, S, CO or COO; u is 0 or 1; Ar⁴ in each occurrence is independently an aromatic or heteroaromatic group, optionally a C₆₋₂₀ aryl, optionally phenyl, which is unsubstituted or substituted with one or more substituents; and v is at least 1.

Substituents of Ar⁴, where present, may be ionic or non-ionic. Exemplary substituents include F, CN, NO₂ and linear, branched or cyclic C₁₋₂₀ alkyl wherein one or more non-adjacent C atoms may be replaced by O, S, NR⁸, CO or COO wherein R⁸ is a C₁₋₁₂ hydrocarbyl and one or more H atoms of the C₁₋₂₀ alkyl may be replaced with F.

Optionally, each Y is S.

The polymer may contain one or more different groups D. Preferably, the polymer contains only one group D.

The repeating structures of formula (I) may all be the same or the polymer may contain two or more different repeating structures of formula (I). Preferably, repeating structure of formula (I) are all the same.

Optionally, the repeating structure of formula (I) is the only repeating structure of the polymer.

The present disclosure provides composition comprising a polymer as described herein and an electron-accepting material.

The present disclosure provides an organic electronic device comprising an active layer comprising compound or composition as described herein.

Optionally, the organic electronic device is an organic photoresponsive device comprising a bulk heterojunction layer comprising the composition as described herein disposed between an anode and a cathode.

Optionally, the organic photoresponsive device is an organic photodetector.

The present disclosure provides a photosensor comprising a light source and an organic photodetector as described herein wherein the photosensor is configured to detect light emitted from a light source.

Optionally, the light source emits light having a peak wavelength of at least 750 nm.

The present disclosure provides a formulation comprising a polymer or a composition as described herein dissolved or dispersed in one or more solvents.

The present disclosure provides a method of forming an organic electronic device according as described herein wherein formation of the active layer comprises deposition of a formulation as described herein onto a surface and evaporation of the one or more solvents.

Optionally, the method comprises polymerising a monomer of formula (VIa) and a monomer of formula (VIb) or polymerising a monomer of formula (VIIa) and a monomer of formula (VIIb):

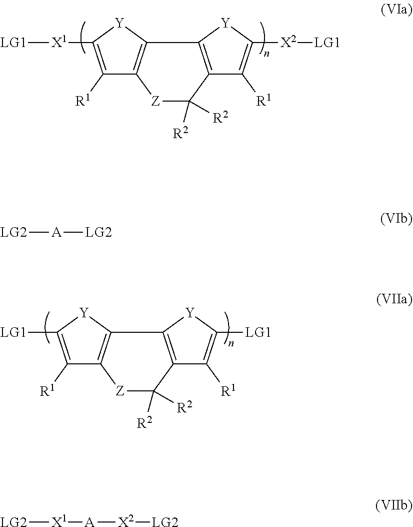

wherein:
R¹, R², n, Y and Z are as described herein;
LG1 is a first leaving group;
LG2 is a second leaving group which is different from LG1; and
a carbon-carbon bond is formed during polymerisation between aromatic carbon atoms of X¹ and X² of formula (VIa) and A of formula (VIb), or between aromatic carbon atoms of formula (VIIa) and X¹ and X² of formula (VIIb).

Optionally, LG1 is selected from one of group (a) and group (b), and LG2 is selected from the other of group (a) and group (b):
(a) halogen or —OSO₂R⁶ wherein R⁶ is an optionally substituted C₁₋₁₂ alkyl group or optionally substituted aryl group;
(b) boronic acid and esters thereof; and —SnR⁹₃ wherein R⁹ independently in each occurrence is a C₁₋₁₂ hydrocarbyl group.

The present disclosure provides compound of formula (VIa):

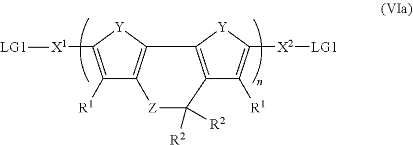

wherein R¹, R², X¹, X², n, Y and Z are as described herein; and LG1 is selected from the group consisting of halogen; —OSO₂R⁶ wherein R⁶ is an optionally substituted C₁₋₁₂ alkyl group or optionally substituted aryl group; boronic acid and esters thereof; and —SnR⁹₃ wherein R⁹ independently in each occurrence is a C₁₋₁₂ hydrocarbyl group.

### DESCRIPTION OF DRAWINGS

The disclosed technology and accompanying figures describe some implementations of the disclosed technology.

FIG. 1 illustrates an organic photoresponsive device according to some embodiments;

FIG. 2 is a graph of current density vs. voltage for an organic photodetector according to some embodiments of the present disclosure and a comparative organic photodetector; and

FIG. 3 is a graph of current density vs. voltage for a comparative organic photodetector.

The drawings are not drawn to scale and have various viewpoints and perspectives. The drawings are some implementations and examples. Additionally, some components and/or operations may be separated into different blocks or combined into a single block for the purposes of discussion of some of the embodiments of the disclosed technology. Moreover, while the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list. References to a layer “over” another layer when used in this application means that the layers may be in direct contact or one or more intervening layers are may be present. References to a layer “on” another layer when used in this application means that the layers are in direct contact. References to a specific atom include any isotope of that atom unless specifically stated otherwise.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

The present inventors have found that donor-acceptor polymers having a strongly electron accepting repeat unit, e.g. for absorption at long wavelengths such as ≥750 nm, may exhibit non-diode behaviour in a device. The present inventors have found that providing a bridging unit between the donor and acceptor repeat units may improve the diode characteristics of a device containing the polymer as compared to a polymer without a bridging unit.

The polymer has a repeating structure of formula (I):

-D-X¹-A-X²- (I)
D is a conjugated electron-donating group of formula (II):

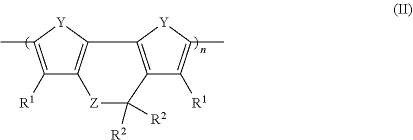

Y in each occurrence is independently O or S, preferably S.
Z is O, S or NR³ wherein R³ is H or a substituent.
R¹ in each occurrence is independently H or a substituent.
R² in each occurrence is independently H or a substituent, preferably a substituent.
n is at least 1, preferably 1, 2 or 3.

The repeating structure of formula (I) is optionally the only repeating structure in the polymer.

Preferably, each R² is independently selected from the group consisting of:

linear, branched or cyclic C₁₋₂₀ alkyl wherein one or more non-adjacent C atoms may be replaced by O, S, NR⁸, CO or COO wherein R⁸ is a C₁₋₁₂ hydrocarbyl and one or more H atoms of the C₁₋₂₀ alkyl may be replaced with F; and
a group of formula (Ak)u—(Ar⁴)v wherein Ak is a C₁₋₁₂ alkylene chain in which one or more C atoms may be replaced with O, S, CO or COO; u is 0 or 1; Ar⁴ in each occurrence is independently an aromatic or heteroaromatic group which is unsubstituted or substituted with one or more substituents; and v is at least 1.

If a C atom of an alkyl group as described anywhere herein is replaced with another atom or group, the replaced C atom may be a terminal C atom of the alkyl group or a non-terminal C-atom. By “non-terminal” C atom of an alkyl group as used herein is meant a C atom of the alkyl other than the methyl C atom of a linear (n-alkyl) chain or the methyl C atoms of a branched alkyl chain.

If a terminal C atom of a group as described anywhere herein is replaced then the resulting group may be an anionic group comprising a countercation, e.g. an ammonium or metal countercation, preferably an ammonium or alkali metal countercation.

A C atom of an alkyl substituent group which is replaced with another atom or group as described anywhere herein is preferably a non-terminal C atom, and the resultant substituent group is preferably non-ionic.

Optionally, each R¹ is independently selected from H and a substituent as described with reference to R². Preferably, each R¹ is H.

Preferably, R³ is a C₁₋₂₀ hydrocarbyl group, optionally a C₁₋₂₀ alkyl; unsubstituted phenyl; or phenyl substituted with one or more C₁₋₁₂ alkyl groups.

Exemplary repeat units of formula (II) include, without limitation:

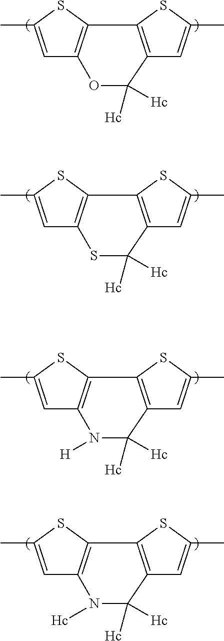

wherein Hc in each occurrence is independently a C₁₋₂₀ hydrocarbyl group, e.g. C₁₋₂₀ alkyl, unsubstituted aryl, or aryl substituted with one or more C₁₋₁₂ alkyl groups. The aryl group is preferably phenyl. In the case where n of formula (II) more than 1, each of then units may be the same or different and each of the n units may be connected in any orientation. For example, when n is 2 the group of formula (II) may be selected from any of:

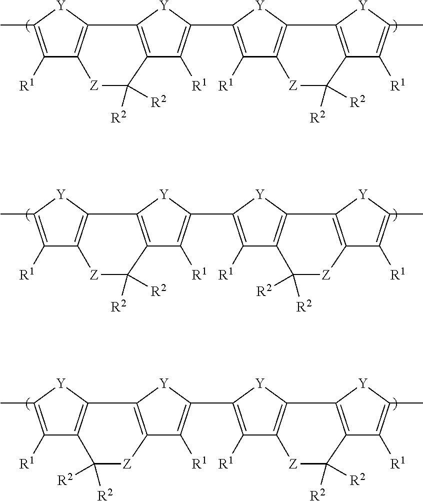

X¹ and X² are the same or different, preferably the same, and in each occurrence is a conjugated bridge group selected from phenylene, thiophene, furan, thienothiophene, furofuran and thienofuran, thiazole, oxazole, alkene, alkyne and imine, preferably thiophene, furan, thienothiophene or furofuran, each of which may be unsubstituted or substituted with one or more substituents. Substituents may be selected from R² groups other than H.

Optionally, X¹ and X² are each independently selected from units of formulae (IIIa)-(IIIg):

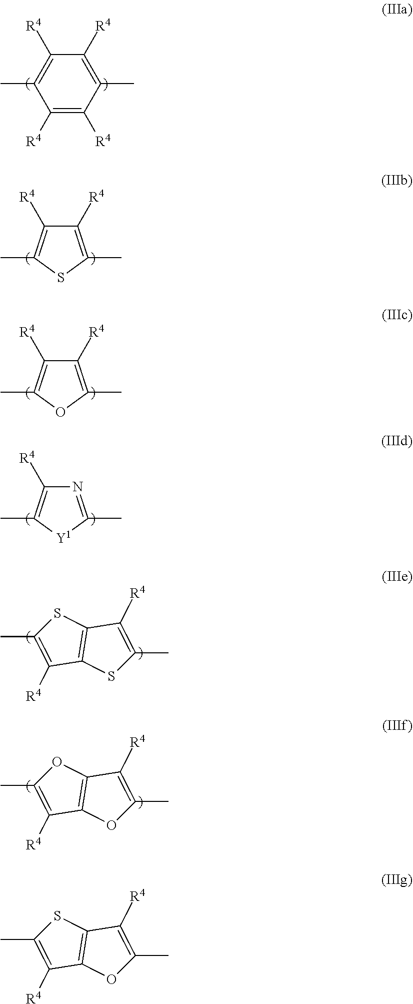

wherein R⁴ in each occurrence is independently H or a substituent and Y¹ is O, S or NR¹¹ wherein R¹¹ is H or a C₁₋₃₀ hydrocarbyl group. Substituents R⁴ may be selected from non-H groups described with respect to R². In some embodiments, a substituent is provided on a carbon atom of X¹ and/or X² which is adjacent to a carbon atom bound directly to electron-donating group D or to electron accepting group A.

The electron-accepting repeat unit A has a LUMO level that is deeper (i.e. further from vacuum) than the LUMO of electron-donating repeat unit D, preferably at least 1 eV deeper. The LUMO levels repeat units of formula (I) and electron-accepting repeat units may be as determined by modelling the LUMO level of each repeat unit, in which bonds to adjacent repeat units are replaced with bonds to a hydrogen atom. Modelling may be performed using Gaussian09 software available from Gaussian using Gaussian09 with B3LYP (functional) and 6-31G (Basis set).

The polymer may have a HOMO of 5.30 eV or shallower, optionally no more than 5.20 eV or no more than 5.10 eV as measured by square wave voltammetry. By “shallower” as used herein in the context of HOMO and LUMO levels is meant closer to vacuum level. Preferably, the polymer has a HOMO in the range of 4.80-5.30 eV.

A model of the polymer of formula H-[D-X¹-A-X²]₂-A modelled as described herein may have a HOMO level of no more than 4.50 eV from vacuum level, preferably no more than 4.40 eV from vacuum level.

Exemplary electron-accepting groups A include, without limitation:

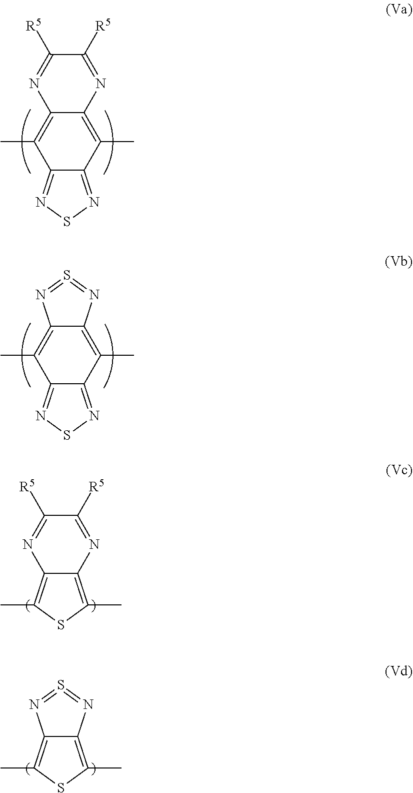

wherein R⁵ in each occurrence is independently H or a substituent, optionally H; F; C₁₋₁₂ alkyl wherein one or more non-adjacent, C atoms may be replaced with O, S, COO or CO and one or more H atoms of the alkyl may be replaced with F; or an aromatic group Ar e , optionally phenyl, which is unsubstituted or substituted with one or more substituents selected from F and C₁₋₁₂ alkyl wherein one or more non-adjacent, C atoms may be replaced with O, S, COO or CO. In the case where one or more C atoms are replaced, the replaced C atom is preferably a non-terminal C atom.

Unless stated otherwise, HOMO and LUMO levels as described herein are as measured by square wave voltammetry.

Exemplary polymers as described herein include polymers wherein the repeating structure of formula (I) are:

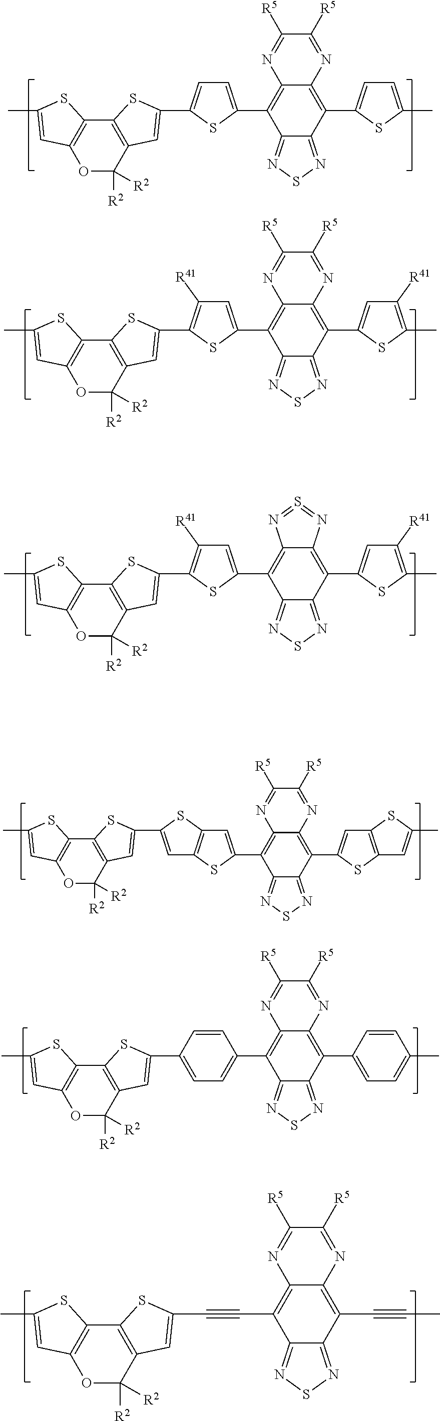

wherein R⁴¹ in each occurrence is independently selected from groups R⁴ other than H.

Preferably, the polystyrene-equivalent number-average molecular weight (Mn) measured by gel permeation chromatography of a polymer as described herein is in the range of about 5×10³ to 1×10⁸, and preferably 1×10⁴ to 5×10⁶. The polystyrene-equivalent weight-average molecular weight (Mw) of the polymer may be 1×10³ to 1×10⁸, and preferably 1×10⁴ to 1×10⁷.

Optionally, the polymer has a HOMO-LUMO band gap of less than 2.00 eV, optionally less than 1.80 eV.

Optionally, the polymer has an absorption spectrum having a peak at a wavelength greater than about 750 nm, optionally in the range of 750-2000 nm. The absorption spectrum may be as measured in solution using a Cary 5000 UV-vis-IR spectrometer.

### Polymer Synthesis and Monomers

A polymer as described herein may be formed by polymerising a monomer for forming electron-donating repeat unit D and electron-accepting repeat unit A wherein one of these monomers further contains groups X¹ and X². The polymerisation method includes, without limitation, methods for forming a carbon-carbon bond between an aromatic carbon atom of an electron-donating unit D and an aromatic carbon atom of an electron-accepting unit A.

In some embodiments, formation of the polymer comprises polymerisation of a monomer of formula (VIa) and a monomer of formula (VIb):

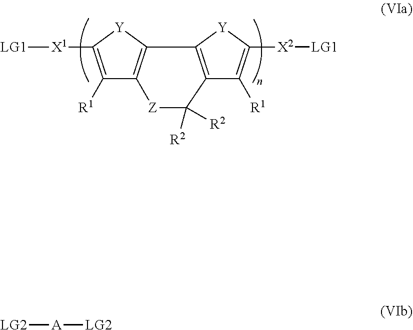

In some embodiments, formation of the polymer comprises polymerisation of a monomer of formula (VIIa) and a monomer of formula (VIIb):

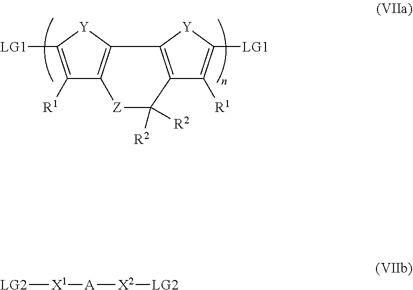

R¹, R², X¹, X², Y and Z are as described above.
LG1 is a first leaving group.
LG2 is a second leaving group which is different from LG1.

A carbon-carbon bond is formed during polymerisation between aromatic carbon atoms of X¹ and X² of formula (VIa) and aromatic carbon atoms of A of formula (VIb), or between aromatic carbon atoms of formula (VIIa) and of X¹ and X² of formula (VIIb).

Optionally, LG1 is selected from one of group (a) and group (b), and LG2 is selected from the other of group (a) and group (b):
(a) halogen or —OSO₂R⁶ wherein R⁶ is an optionally substituted C₁₋₁₂ alkyl group or optionally substituted aryl group;
(b) boronic acid and esters thereof; and —SnR⁹₃ wherein R⁹ independently in each occurrence is a C₁₋₁₂ hydrocarbyl group.

Suitable polymerisation methods include, without limitation, Suzuki polymerisation and Stille polymerisation. Suzuki polymerisation is described in, for example, WO 00/53656.

In some embodiments, each LG1 may be one of: (i) a halogen or —OSO₂R⁶; or (ii), a boronic acid or ester, and each LG2 may be the other of (i) and (ii).

In some embodiments, each LG1 may be one of: (i) a halogen or —OSO₂R⁶; and (iii) —SnR⁹₃, and each LG2 may be the other of (i) and (iii).

Optionally, R⁶ in each occurrence is independently a C₁₋₁₂ alkyl group which is unsubstituted or substituted with one or more F atoms; or phenyl which is unsubstituted or substituted with one or more F atoms.

—OSO₂R⁶ is preferably tosylate or triflate.

Exemplary boronic esters have formula (VIII):

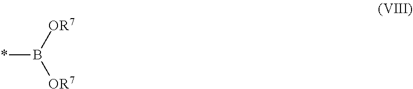

wherein R⁷ in each occurrence is independently a C₁₋₂₀ alkyl group in which non-adjacent C atoms may be replaced with O, C═O or NR¹⁰ wherein R¹⁰ is a C₁₋₁₂ alkyl, * represents the point of attachment of the boronic ester to an aromatic ring of the monomer, and the two groups R⁷ may be linked to form a ring which is unsubstituted or substituted with one or more substituents, e.g. one or more C₁₋₆ alkyl groups.

Optionally, R⁷ independently in each occurrence is selected from the group consisting of C₁₋₁₂ alkyl; unsubstituted phenyl; and phenyl substituted with one or more C₁₋₆ alkyl groups.

In a preferred embodiment, the two groups R⁷ are linked, e.g. to form:

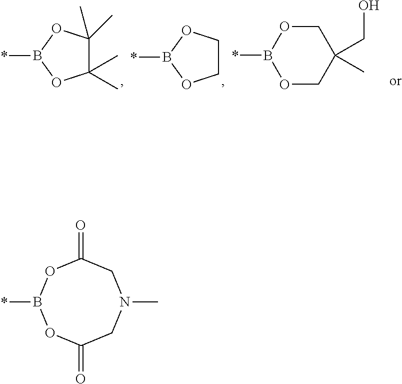

A halogen leaving group is preferably Br or I.

### Compositions

The polymer may be part of a composition comprising or consisting of an electron-accepting (n-type) material and an electron-donating (p-type) material wherein the polymer is the electron-donating material. The composition may comprise one or more further materials, e.g. one or more further electron-donating materials and/or one or more further electron-accepting materials.

In some embodiments, the weight ratio of the electron donor material(s) comprising or consisting of a polymer as described herein to the acceptor material(s) is from about 1:0.5 to about 1:2, preferably about 1:1.1 to about 1:2.

The electron-accepting material has a LUMO level that is deeper (i.e. further from vacuum) than the LUMO of the electron-donating polymer. Optionally, the gap between the HOMO level of the electron-donating polymer and the LUMO level of the electron-accepting material is less than 1.4 eV. Unless stated otherwise, HOMO and LUMO levels of materials as described herein are as measured by square wave voltammetry (SWV).

The, or each, electron acceptor material is preferably a non-polymeric compound. Preferably, the non-polymeric compound has a molecular weight of less than 5,000 Daltons, optionally less than 3,000 Daltons.

The electron acceptor material may be a fullerene or a non-fullerene

Non-fullerene acceptors are described in, for example, Cheng et al, “Next-generation organic photovoltaics based on non-fullerene acceptors”, Nature Photonics volume 12, pages 131-142 (2018), the contents of which are incorporated herein by reference, and which include, without limitation, PDI, ITIC, ITIC, IEICO and derivatives thereof, e.g. fluorinated derivatives thereof such as ITIC-4F and IEICO-4F.

Exemplary fullerene electron acceptor materials are C₆₀, C₇₀, C₇₆, C₇₈ and C₈₄ fullerenes or a derivative thereof including, without limitation, PCBM-type fullerene derivatives (including phenyl-C61-butyric acid methyl ester (C₆₀PCBM), TCBM-type fullerene derivatives (e.g. tolyl-C61-butyric acid methyl ester (C₆₀TCBM)), and ThCBM-type fullerene derivatives (e.g. thienyl-C61-butyric acid methyl ester (C₆₀ThCBM).

### Organic Electronic Devices

A polymer or composition as described herein may be provided as an active layer of an organic electronic device. In a preferred embodiment, a bulk heterojunction layer of an organic photoresponsive device, more preferably an organic photodetector, comprises a composition as described herein.

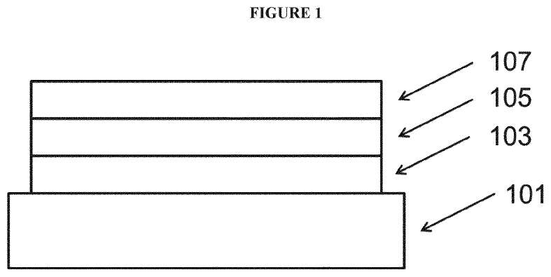

FIG. 1 illustrates an organic photoresponsive device according to some embodiments of the present disclosure. The organic photoresponsive device comprises a cathode 103, an anode 107 and a bulk heterojunction layer 105 disposed between the anode and the cathode. The organic photoresponsive device may be supported on a substrate 101, optionally a glass or plastic substrate.

Each of the anode and cathode may independently be a single conductive layer or may comprise a plurality of layers.

At least one of the anode and cathode is transparent so that light incident on the device may reach the bulk heterojunction layer. In some embodiments, both of the anode and cathode are transparent.

Each transparent electrode preferably has a transmittance of at least 70%, optionally at least 80%, to wavelengths in the range of 750-1800 nm. The transmittance may be selected according to an emission wavelength of a light source for use with the organic photodetector.

FIG. 1 illustrates an arrangement in which the cathode is disposed between the substrate and the anode. In other embodiments, the anode may be disposed between the cathode and the substrate.

The organic photoresponsive device may comprise layers other than the anode, cathode and bulk heterojunction layer shown in FIG. 1. In some embodiments, a hole-transporting layer is disposed between the anode and the bulk heterojunction layer. In some embodiments, an electron-transporting layer is disposed between the cathode and the bulk heterojunction layer.

In some embodiments, a work function modification layer is disposed between the bulk heterojunction layer and the anode, and/or between the bulk heterojunction layer and the cathode.

The area of the OPD may be less than about 3 cm², less than about 2 cm², less than about 1 cm², less than about 0.75 cm², less than about 0.5 cm² or less than about 0.25 cm². Optionally, each OPD may be part of an OPD array wherein each OPD is a pixel of the array having an area as described herein, optionally an area of less than 1 mm², optionally in the range of 0.5 micron²-900 micron².

The substrate may be, without limitation, a glass or plastic substrate. The substrate can be an inorganic semiconductor. In some embodiments, the substrate may be silicon. For example, the substrate can be a wafer of silicon. The substrate is transparent if, in use, incident light is to be transmitted through the substrate and the electrode supported by the substrate.

The bulk heterojunction layer contains a polymer as described herein and an electron acceptor material. The bulk heterojunction layer may consist of these materials or may comprise one or more further materials, for example one or more further electron donor materials and/or one or more further electron acceptor materials.

### Formulations

A layer containing a polymer or composition as described herein may be formed by depositing a formulation containing a polymer or a composition as described herein dissolved or dispersed in one or more solvents and evaporating the one or more solvents.

The formulation may be deposited by any coating or printing method including, without limitation, spin-coating, dip-coating, roll-coating, spray coating, doctor blade coating, wire bar coating, slit coating, ink jet printing, screen printing, gravure printing and flexographic printing.

The one or more solvents of the formulation may optionally comprise or consist of benzene substituted with one or more substituents selected from chlorine, C₁₋₁₀ alkyl and C₁₋₁₀ alkoxy wherein two or more substituents may be linked to form a ring which may be unsubstituted or substituted with one or more C₁₋₆ alkyl groups, optionally toluene, xylenes, trimethylbenzenes, tetramethylbenzenes, anisole, indane and its alkyl-substituted derivatives, and tetralin and its alkyl-substituted derivatives.

The formulation may comprise a mixture of two or more solvents, preferably a mixture comprising at least one benzene substituted with one or more substituents as described above and one or more further solvents. The one or more further solvents may be selected from esters, optionally alkyl or aryl esters of alkyl or aryl carboxylic acids, optionally a C₁₋₁₀ alkyl benzoate, benzyl benzoate or dimethoxybenzene.

The formulation may comprise further components. As examples of such components, adhesive agents, defoaming agents, deaerators, viscosity enhancers, diluents, auxiliaries, flow improvers colorants, dyes or pigments, sensitizers, stabilizers, nanoparticles, surface-active compounds, lubricating agents, wetting agents, dispersing agents and inhibitors may be mentioned.

### Applications

A circuit may comprise an organic photodetector as described herein connected to a voltage source for applying a reverse bias to the device and/or a device configured to measure photocurrent. The voltage applied to the photodetector may be variable. In some embodiments, the photodetector may be continuously biased when in use.

In some embodiments, a photodetector system comprises a plurality of photodetectors as described herein, such as an image sensor of a camera.

In some embodiments, a sensor may comprise an OPD as described herein and a light source wherein the OPD is configured to receive light emitted from the light source. In some embodiments, the light source has a peak wavelength of at least 750 nm.

In some embodiments, the light from the light source may or may not be changed before reaching the OPD. For example, the light may be reflected, filtered, down-converted or up-converted before it reaches the OPD.

The organic photoresponsive device as described herein may be an organic photovoltaic device or an organic photodetector. An organic photodetector as described herein may be used in a wide range of applications including, without limitation, detecting the presence and/or brightness of ambient light and in a sensor comprising the organic photodetector and a light source. The photodetector may be configured such that light emitted from the light source is incident on the photodetector and changes in wavelength and/or brightness of the light may be detected, e.g. due to absorption by, reflection by and/or emission of light from an object, e.g. a target material in a sample disposed in a light path between the light source and the organic photodetector. The sensor may be, without limitation, a gas sensor, a biosensor, an X-ray imaging device, an image sensor such as a camera image sensor, a motion sensor (for example for use in security applications) a proximity sensor or a fingerprint sensor. A 1D or 2D photosensor array may comprise a plurality of photodetectors as described herein in an image sensor.

### EXAMPLES

### Monomer 1

Monomer 1 was prepared according to the following reaction scheme:

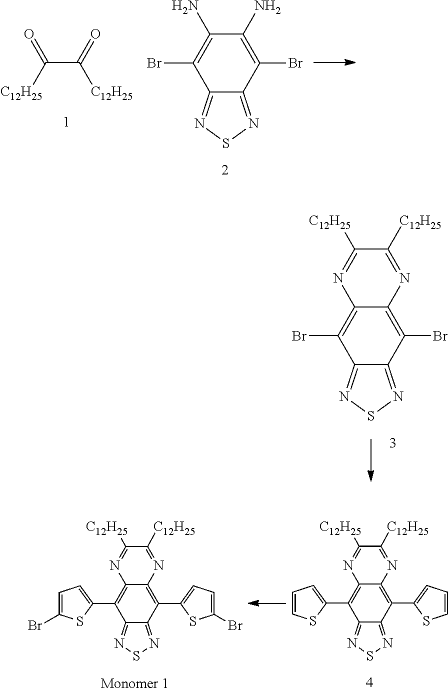

### Monomer 1 Stage 1

Compound 1 was synthesized as described in J. Org. Chem., 2002, 67, 9073, the contents of which are incorporated herein by reference.

Compound 2 was synthesized as described in J. Org. Chem., 2017, 82, 3132, the contents of which are incorporated herein by reference.

1 (30 g, 92 mmol) was taken in acetic acid (2.5 L) and purged with nitrogen. 2 (73.1 g, 186 mmol) was added portions wise and then the reaction mixture was heated to 40° C. for 16 h. Water was added and the mixture was stirred for 0.5 h and filtered. The solid was dissolved in DCM (1.5 L) and washed with water (3×2 L), dried, filtered and concentrated. The crude product was further purified by column chromatography (silica, ethyl acetate in hexanes as elutant) to give 3 (48 g, 46%) with >96% purity as measured by HPLC.

### Monomer 1 Stage 2

Bistriphenylphosphine palladiumdichloride (5 mol %) was added to a nitrogen-purged solution of 3 (50 g, 73.3 mmol) and thiophene-2-tributyltin (68.4 g, 183 mmol) in dry toluene (1 L) and the reaction stirred at 75° C. overnight. A further 2 mol % catalyst was added, and the reaction was stirred at 80° C. overnight. The mixture was cooled and filtered through celite eluting with toluene. The solvent was removed to yield the crude product which was further purified by precipitation from DCM/methanol. The resulting solid was triturated with ethyl acetate and filtered before being dissolved in toluene and crystallised at −40° C. The isolate solid was filtered to give 4 (40 g, 79%) with >99% purity as measured by HPLC.

### Monomer 1

A solution of N-bromosuccinimide (13.35 g, 75 mmol) in nitrogen-purged DMF (100 mL) was added dropwise to a nitrogen-purged solution of 4 (35 g, 50 mmol) in chloroform (1 L) at −40° C. and the reaction mixture stirred overnight. After this time, the reaction mixture was again cooled to −40° C. and further portions of N-bromosuccinimide in nitrogen-purged DMF were added until LC analysis showed >90% of the product (a total 18.15 g of N-bromosuccinimide was added). Chloroform (500 mL) and water (1 L) were then added, the layers separated and the organic phase washed with water (2×1.5 L), dried with sodium sulfate, filtered and concentrated. The crude product was purified by column chromatography (silica, DCM in hexanes and then ethyl acetate in hexanes as elutants). Fractions containing the product were recrystallized from toluene/ethyl acetate and triturated from acetone to give Monomer 1 (26.7 g, 62%) with >99% purity as measured by HPLC.

### Polymerisation

Exemplary and comparative polymers were prepared as described in US2016372675, the contents of which are incorporated herein by reference.

In preparation of the example and comparative polymers, R² of 50 mol % of the monomers for forming the donor repeat units is C₁₂H₂₅ and R² of the other 50 mol % of the monomers for forming the donor repeat units is 3,7-dimethyloctyl:

### HOMO and LUMO Measurements

HOMO and LUMO values of polymer films were measured by square wave voltammetry.

In square wave voltammetry, the current at a working electrode is measured while the potential between the working electrode and a reference electrode is swept linearly in time. The difference current between a forward and reverse pulse is plotted as a function of potential to yield a voltammogram. Measurement may be with a CHI 660D Potentiostat.

The apparatus to measure HOMO or LUMO energy levels by SWV may comprise a cell containing 0.1 M tertiary butyl ammonium hexafluorophosphate in acetonitrile; a 3 mm diameter glassy carbon working electrode; a platinum counter electrode and a leak free Ag/AgCl reference electrode.

Ferrocene is added directly to the existing cell at the end of the experiment for calculation purposes where the potentials are determined for the oxidation and reduction of ferrocene versus Ag/AgCl using cyclic voltammetry (CV).

The sample is dissolved in Toluene (3 mg/ml) and spun at 3000 rpm directly on to the glassy carbon working electrode.

LUMO=4.8−E ferrocene (peak to peak average)−E reduction of sample (peak maximum).

HOMO=4.8−E ferrocene (peak to peak average)+E oxidation of sample (peak maximum).

A typical SWV experiment runs at 15 Hz frequency; 25 mV amplitude and 0.004 V increment steps. Results are calculated from 3 freshly spun film samples for both the HOMO and LUMO data.

Table 1 contains HOMO and LUMO values for Polymer Example 1 which contains bridging thiophene units and Comparative Polymers 1-3 which do not contain bridging thiophene units.

| Polymer | HOMO (eV) | LUMO (eV) | Band gap (eV) |
|---|---|---|---|
| Polymer Example 1 | −5.05 | −3.45 | 1.60 |
| Comparative Polymer 1 | −4.79 | −3.72 | 1.07 |
| Comparative Polymer 2 | −4.77 | −3.48 | 1.29 |
| Comparative Polymer 3 | −5.28 | 3.05 (peak apex) | 2.23 |
| | | 3.11 (shoulder) | |

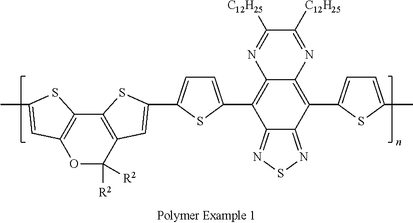

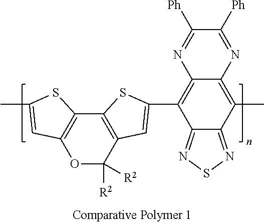

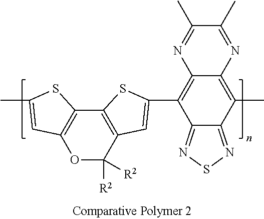

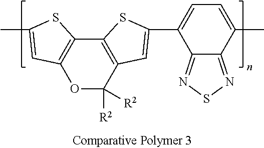

WO 2022/090522 PCT/EP2021/080222

In Polymer Example 1, Comparative Polymer 1 and Comparative Polymer 2, R² is 3,7-dimethyloctyl for 50% of n and is C₁₂H₂₅ for the other 50%.

In Comparative Polymer 3, all R² groups are 3,7-dimethyloctyl.

### Photodetector Example 1

A device having the following structure was prepared:

Cathode/Donor:Acceptor layer/Anode

A glass substrate coated with a layer of indium-tin oxide (ITO) was treated with polyethyleneimine (PEIE) to modify the work function of the ITO.

A mixture of Polymer Example 1 (donor) and C₆₀PCBM (acceptor) in a donor : acceptor mas ratio of 1:1.75 was deposited over the modified ITO layer by bar coating from a 15 mg/ml solution in 1,2,4 Trimethylbenzene; 1,2-Dimethoxybenzene 95:5 v/v solvent mixture. The film was dried at 80° C. to form a ca. 500 nm thick bulk heterojunction layer

An anode stack of MoO₃ (10 nm) and ITO (150 nm) was formed over the bulk heterojunction by thermal evaporation (MoO₃) and sputtering (ITO).

### Comparative Photodetector 1

A device was prepared as described for Photodetector Example 1 except that Comparative Polymer 2 was used in place of Polymer Example 1.

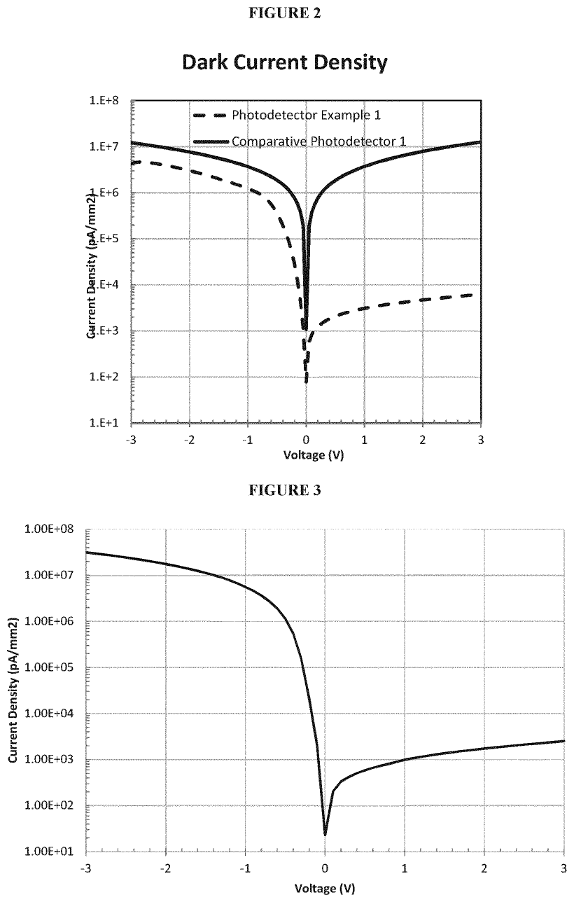

Dark current (i.e. current upon application of a bias in the absence of any incident light) of Comparative Photodetector 1 and Photodetector Example 1 were measured. With reference to FIG. 2, Photodetector Example 1 demonstrates diode-like behaviour upon application of a bias whereas Comparative Photodetector 1 does not. Without wishing to be bound by any theory, it is believed that doping occurs upon exposure of the donor polymer.

### Comparative Photodetector 2

A device was prepared as described for Photodetector Example 1 except that Comparative Polymer 3 was used in place of Polymer Example 1 and in that the anode was formed over the bulk heterojunction layer by spin-coating Clevios HIL-E100.

With reference to FIG. 3, Comparative Photodetector 2 demonstrates diode-like behaviour. Without wishing to be bound by any theory, it is believed that there is little or no doping of the polymer due to its relatively deep HOMO.

### Quantum Chemical Modelling Examples

All modelling as described in these examples was performed using software available from Gaussian using Gaussian09 with B3LYP (functional).

HOMO and LUMO levels of acceptor groups A were modelled and the results are set out in Table 1.

TABLE 1

| | A HOMO/ | A LUMO/ |
|---|---|---|
| A Structure | eV | eV |
| 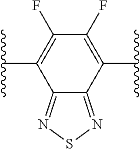 [SMILES: *c1c(F)c(F)c(*)c2nsnc12] | −6.95 | −2.60 |
| 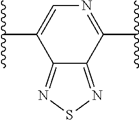 [SMILES: *c1cnc(*)c2nsnc12] | −7.09 | −2.82 |
| 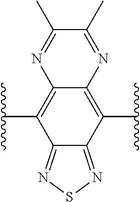 [SMILES: *c1c2nsnc2c(*)c2nc(C)c(C)nc12] | −6.17 | −2.93 |
| 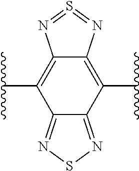 [SMILES: *c1c2c(c(*)c3nsnc13)N=S=N2] | −6.20 | −3.56 |
| 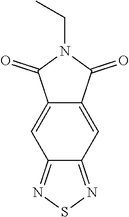 [SMILES: CCN1C(=O)c2cc3nsnc3cc2C1=O] | −7.27 | −3.03 |
| 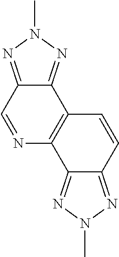 [SMILES: Cn1nc2cnc3c(ccc4nn(C)nc43)c2n1] | −6.06 | −1.83 |

HOMO and LUMO levels of donor units and co-repeat units were modelled and the results are set out in Table 2.

TABLE 2

| | A HOMO/ | A LUMO/ |
|---|---|---|
| Structure | eV | eV |
| 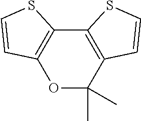 | −5.08 | −1.09 |
| 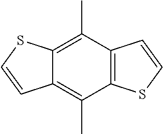 | −5.26 | −0.99 |
| 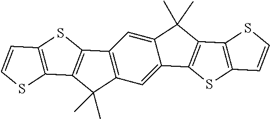 [SMILES: CC1(C)c2cc3c(cc2-c2sc4ccsc4c21)C(C)(C)c1c-3sc2ccsc12] | −4.88 | −1.51 |
| 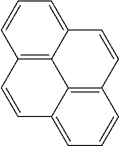 [SMILES: c1cc2ccc3cccc4ccc(c1)c2c34] | −5.32 | −1.48 |

HOMO and LUMO levels of comparative and exemplary compounds were modelled and the results are set out in Table 3.

TABLE 3

| | | QC | QC |
|---|---|---|---|
| | | HOMO | LUMO |
| Name | Structure | (eV) | (eV) |
| Comparative Structure 1 | 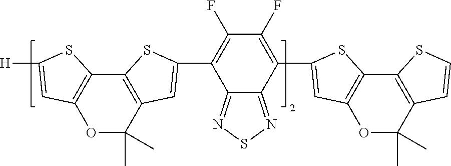 | −4.65 | −2.92 |
| Comparative Structure 2 | 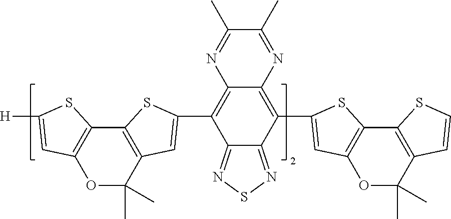 | −4.25 | −3.21 |
| Comparative Structure 3 | 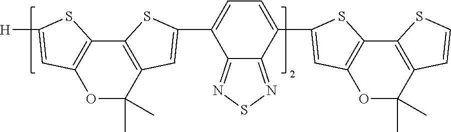 | −4.49 | −2.81 |
| Comparative Structure 4 | 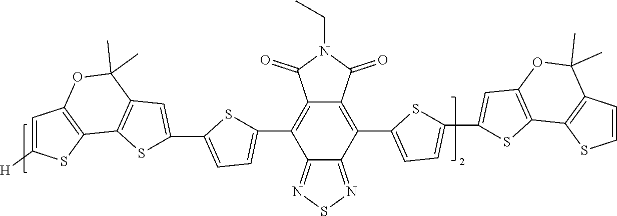 | −4.73 | −2.99 |
| Comparative Structure 5 | 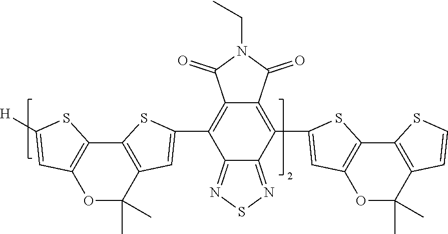 | −4.83 | −3.01 |
| Comparative Structure 6 | 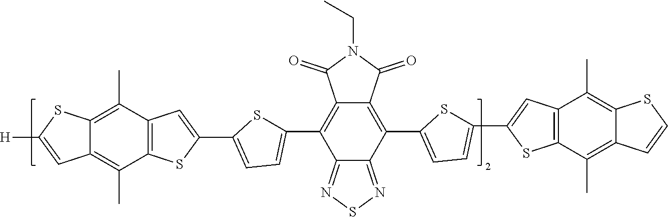 | −5.04 | −3.05 |
| Comparative Structure 7 | 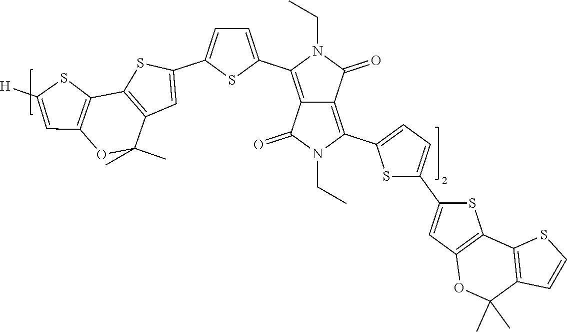 [SMILES: CCN1C(=O)C2=C(c3ccc(-c4cc5c(s4)-c4sccc4OC5(C)C)s3)N(CC)C(=O)C2=C1c1ccc(-c2cc3c(s2)-c2sccc2C(C)(C)O3)s1] | −4.53 | −2.77 |
| Comparative Structure 8 | 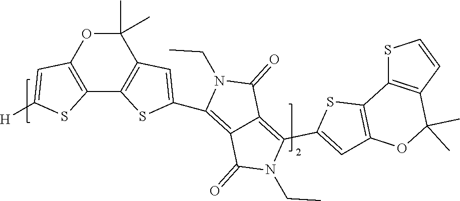 | −4.49 | −2.77 |
| Comparative Structure 9 | 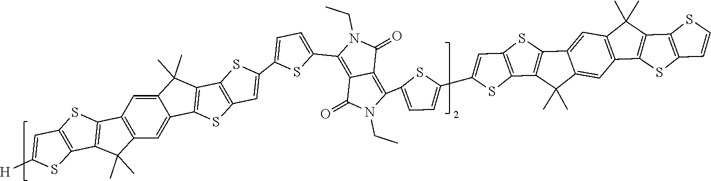 | −4.57 | −2.70 |
| Comparative Structure 10 | 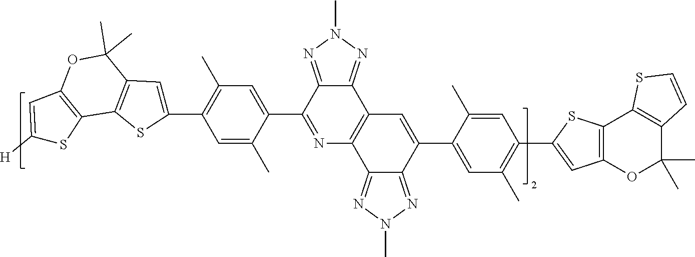 | −4.75 | −2.07 |
| Example Structure 1 | 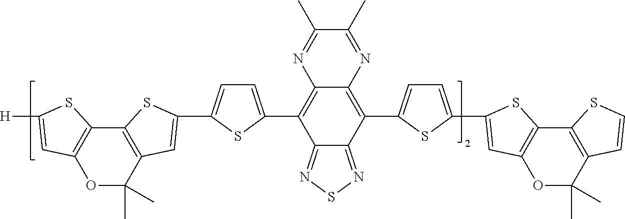 | −4.31 | −3.19 |
| Example Structure 2 | 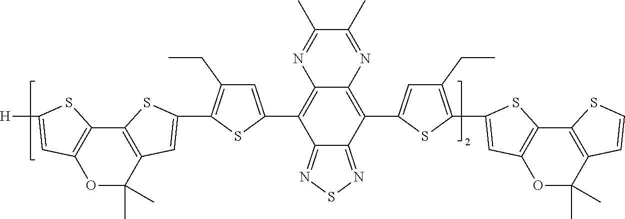 | −4.29 | −3.08 |
| Example Structure 3 | 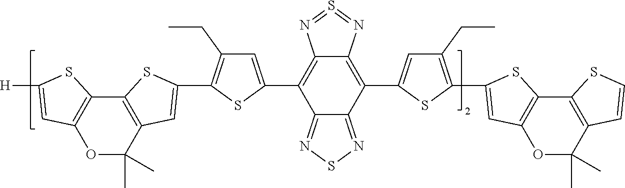 | −4.30 | −3.44 |
| Example Structure 4 | 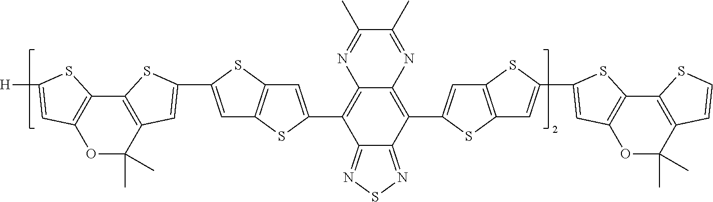 | −4.41 | −3.22 |
| Example Structure 5 | 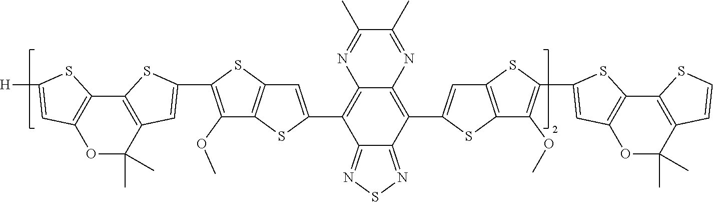 | −4.28 | −3.25 |
| Example Structure 6 | 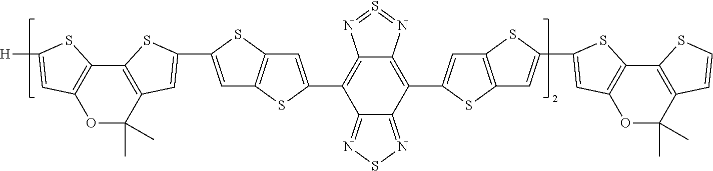 | −4.41 | −3.56 |

WO 2022/090522 PCT/EP2021/080222

As shown in Table 3, materials containing bridging units have a deeper HOMO than comparative materials without bridging units.

1. A polymer comprising a repeating structure of formula (I):
-D-X¹-A-X²- (I)
wherein:
D is a conjugated electron-donating group of formula (II); A is a conjugated electron-accepting group; X¹ and X² are each independently a conjugated bridge group selected from phenylene, thiophene, furan, thienothiophene, furofuran, thienofuran, thiazole, oxazole, alkene, alkyne and imine, each of which may be unsubstituted or substituted with one or more substituents: 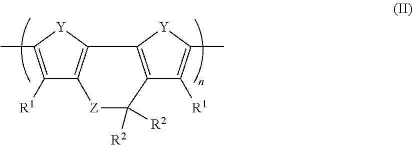
wherein:
Y in each occurrence is independently O or S;
Z is O, S or NR³ wherein R³ is H or a substituent;
R¹ in each occurrence is independently H or a substituent;
R² in each occurrence is independently a substituent,
n is at least 1; and
wherein the polymer has a highest occupied molecular orbital (HOMO) level as measured by square wave voltammetry of no more than 5.30 eV from vacuum level.

2. A polymer comprising a repeating structure of formula (I):
-D-X¹-A-X²- (I)
wherein:
D is a conjugated electron-donating group of formula (II); A is a conjugated electron-accepting group; X¹ and X² are each independently a conjugated bridge group selected from phenylene, thiophene, furan, thienothiophene, furofuran, thienofuran, thiazole, oxazole, alkene, alkyne and imine, each of which may be unsubstituted or substituted with one or more substituents: 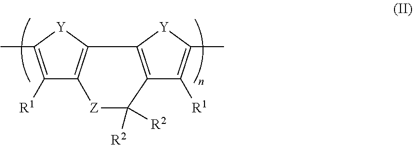
wherein:
Y in each occurrence is independently O or S;
Z is O, S or NR³ wherein R³ is H or a substituent;
R¹ in each occurrence is independently H or a substituent;
R² in each occurrence is independently a substituent,
n is at least 1; and
wherein a model of the polymer of formula H-[D-X¹-A-X²]₂-A has a highest occupied molecular orbital (HOMO) level as modeled using Gaussian09 with B3LYP (functional) and 6-31G (Basis set) of no more than 4.50 eV from vacuum level.

3. The polymer according to claim 1 wherein the polymer has a HOMO level of no more than 5.10 eV from vacuum level.

4. The polymer according to claim 1 wherein the polymer has a HOMO level of at least 4.90 eV from vacuum level as measured by square wave voltammetry.

5. The polymer according to claim 1 wherein the electron-accepting repeat unit is selected from formulae (Va) and (Vb): 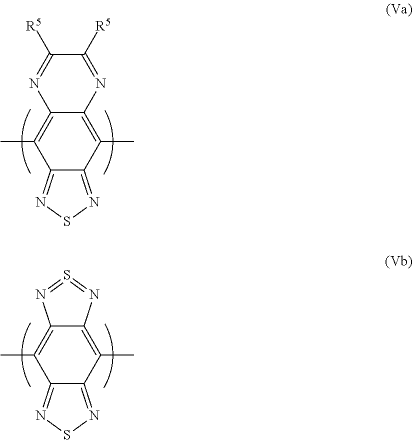
wherein R⁵ in each occurrence is H or a substituent.

6. The polymer according to claim 1 wherein each R¹ is H.

7. The polymer according to claim 1 wherein each R² is independently selected from the group consisting of:
linear, branched or cyclic C₁₋₂₀ alkyl wherein one or more non-adjacent C atoms may be replaced by O, S, NR⁸, CO or COO wherein R⁸ is a C₁₋₁₂hydrocarbyl and one or more H atoms of the C₁₋₂₀ alkyl may be replaced with F; and
a group of formula (Ak)u—(Ar⁴)v wherein Ak is a C₁₋₁₂ alkylene chain in which one or more C atoms may be replaced with O, S, CO or COO; u is 0 or 1; Ar⁴ in each occurrence is independently an aromatic or heteroaromatic group which is unsubstituted or substituted with one or more substituents; and v is at least 1.

8. The polymer according to claim 1 wherein each Y is S.

9. The polymer according to claim 1 wherein X¹ and X² are each independently selected from thiophene, furan, thienothiophene, furofuran and thienofuran each of which is unsubstituted or substituted with one or more substituents.

10. The polymer according to claim 1 wherein repeating structure of formula (I) is the only repeating structure of the polymer.

11. A composition comprising a polymer according to claim 1 and an electron-accepting material.

12. An organic electronic device comprising an active layer comprising a polymer according to claim 1.

13. An organic electronic device according to claim 12 wherein the organic electronic device is an organic photoresponsive device comprising a bulk heterojunction layer comprising the polymer comprising a repeating structure of formula (I) and an electron-accepting material disposed between an anode and a cathode.

14. An organic electronic device according to claim 13 wherein the organic photoresponsive device is an organic photodetector.

15. A photosensor comprising a light source and an organic photodetector according to claim 14 wherein the photosensor is configured to detect light emitted from a light source.

16. A photosensor according to claim 15 wherein the light source emits light having a peak wavelength of at least 750 nm.

17. A formulation comprising a polymer according to claim 1 dissolved or dispersed in one or more solvents.

18. A method of forming an organic electronic device according to claim 12 wherein formation of the active layer comprises deposition of a formulation comprising a polymer comprising a repeating structure of formula (I) dissolved or dispersed in one or more solvents onto a surface and evaporation of the one or more solvents.

19. A method of forming a polymer according to claim 1, the method comprising polymerising a monomer of formula (VIa) and a monomer of formula (VIb) or polymerising a monomer of formula (VIIa) and a monomer of formula (VIIb): 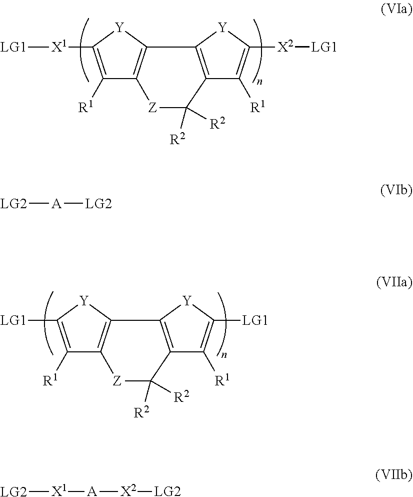
wherein:
R¹, R², n, Y and Z are as defined in any one of claims 1-7;
LG1 is a first leaving group;
LG2 is a second leaving group which is different from LG1; and
a carbon-carbon bond is formed during polymerisation between aromatic carbon atoms of X¹ and X² of formula (VIa) and A of formula (VIb), or between aromatic carbon atoms of formula (VIIa) and X¹ and X² of formula (VIIb).

20. The method according to claim 19 wherein LG1 is selected from one of group (a) and group (b), and LG2 is selected from the other of group (a) and group (b):
a. halogen or —OSO₂R⁶ wherein R⁶ is an optionally substituted C₁₋₁₂ alkyl group or optionally substituted aryl group;
b. boronic acid and esters thereof; and —SnR⁹₃ wherein R⁹ independently in each occurrence is a C₁₋₁₂ hydrocarbyl group.

21. A compound of formula (VIa): 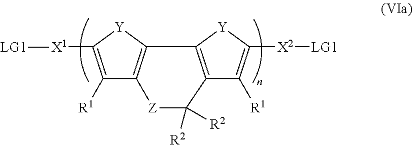
wherein R¹, R², X¹, X², n, Y and Z are as defined in claim 1; and LG1 is selected from the group consisting of halogen; —OSO₂R⁶ wherein R⁶ is an optionally substituted C₁₋₁₂ alkyl group or optionally substituted aryl group; boronic acid and esters thereof; and —SnR⁹₃ wherein R⁹ independently in each occurrence is a C₁₋₁₂ hydrocarbyl group.
